(19) United States
(12) Patent Application Publication
(10) Pub. No.: US 2009/0062802 A1
(43) Pub. Date: Mar. 5, 2009

(54) CUTTING APPARATUS

(75) Inventors: Shaun Palmer, Chapeltown (GB); Gursharan Singh Chana, Sutton Coldfield (GB)

Correspondence Address: PATENTTM.US, P. O. BOX 82788, PORTLAND, OR, 97282-0788, US

(73) Assignee: COMIS ORTHOPAEDICS LIMITED, Templeborough (GB)

(21) Appl. No.: 12/164,496

(22) Filed: Jun. 30, 2008

(30) Foreign Application Priority Data
Aug. 31, 2007 (GB) 0716837.0
Nov. 14, 2007 (GB) 0722308.4

(51) Int. Cl. A61B 17/00; A61B 17/32

(52) U.S. Cl. 606/79; 606/171; 606/170

(57) ABSTRACT

A cutting apparatus comprises a flexible elongate cutting member, and a tensioning arrangement to maintain the flexible elongate member in a tensioned condition. The tensioning arrangement comprises a carrying arrangement on which the flexible elongate member is arranged. The cutting apparatus further includes a driving arrangement to move the carrying arrangement, thereby causing the flexible elongate member to move and effect cutting of an article.

This invention relates to cutting apparatus. More particularly, but not exclusively, this invention relates to cutting apparatus for the removal of stems of implants from bones.

In orthopaedic surgery, it is often necessary to remove an implant that was implanted in an earlier operation. This can be necessary if, for example, the implant has become worn or damaged during use. Where the implant used a hydroxyapatite coating, it can often be the case that the bone material has merged and bonded with the hydroxyapatite. In such cases, it can be exceedingly difficult to remove the stem from the bone.

According to one aspect of this invention, there is provided a cutting apparatus comprising a flexible elongate cutting member, and a tensioning arrangement to maintain the flexible elongate member in a tensioned condition, the tensioning arrangement comprising a carrying arrangement on which the flexible elongate member is arranged, wherein the carrying arrangement is driveable, thereby causing the flexible elongate member to move and effect cutting of an article.

The flexible elongate member may be wound around the carrying arrangement. The carrying arrangement may comprise a rotary arrangement, and the flexible elongate member may be wound around the rotary arrangement. The rotary arrangement may comprise a rotary member and at least one bearing on which the rotary member is mounted to allow rotation of the rotary member.

In one embodiment, the rotary arrangement may comprise first and second bearings, which may be provided at respective opposite ends of the rotary member. The rotary member may be rotatable on the bearings.

A driving element may be connected to the rotary member to effect the aforesaid movement of the carrying arrangement. The driving element may have a connecting portion configured to be connected to a prime mover, such as a driving tool, which may be a power tool.

The tensioning arrangement may further comprise at least one roller member, around which the elongate member may pass. In one embodiment, the tensioning arrangement may comprise first and second of the aforesaid roller members. The, or each, roller member may comprise respective first and second jockey wheels.

The, or each, roller member may be movable between a tension position in which the flexible elongate member is held in tension, and a release position, in which the tension in the flexible elongate member is relaxed. When the, or each, roller member is in the release position, a desired length of the flexible elongate member may be fed from the rotary arrangement and a length less than said desired length is fed onto the rotary arrangement at the same time.

The tensioning arrangement may comprise urging means to urge the, or each, roller member to the tension condition. The urging means may comprise a resilient urging member, such as a spring, conveniently a tension spring. The urging means may comprise two of the aforesaid resilient urging members.

A housing may be provided to house the tensioning arrangement. The housing may be movable relative to the rotary member to move the, or each, roller member between the tension and the release positions.

The housing may be pivotally movable about the rotary member. The movement of the housing may move the roller members between the tension position and the release position.

The housing may be connected on one side of the rotary member to the urging means. A connecting rod may extend across the housing, and the urging means may be attached to the connecting rod.

The, or each, roller member may be rotatably mounted on the housing. The, or each, roller member may be mounted on the housing on the opposite side of the rotary member to the urging means.

A release means may be provided to move the, or each, roller member to the release position. The release means may be mounted on the housing to pivot the housing about the rotary member to the release position. The release means may be pivotally attached to the housing.

The release means may comprise a manipulating element, such as a handle or a knob, and may also comprise a connecting lever. The connecting lever may be pivotally attached to the housing. The connecting lever may be connected to the housing by being attached to the connecting rod. The connecting lever may be pivotally connected to the manipulating element.

The first and second roller members may be rotatable in opposite directions. The first and second roller members may be freely rotatable about their axes.

The tensioning arrangement may further comprise a holding arrangement around the rotary member to hold the flexible elongate member on the rotary member.

The holding arrangement may comprise a housing which may be closely spaced from the rotary member to allow rotation of the rotary member relative thereto, and may be close enough to the rotary member to prevent uncoiling of the flexible elongate member from the rotary member when the roller members are in the release position.

The holding arrangement may define first and second apertures to allow the elongate member to be fed to or from the drum. The first and second apertures may be slots.

A guide arrangement may be provided to guide the flexible elongate member. The guide arrangement may comprise first and second guide conduits along which the elongate member may travel. The elongate flexible member may extend through the guide conduits.

The guide conduits may comprise at least one guide tube. The, or each, guide tube may be formed of a rigid material to allow the elongate member to be pushed into the material to be cut.

The, or each, guide tube may comprise a cable housing, which may comprise an elongate tubular housing. In one embodiment, the guide arrangement may comprise a pair of guide tubes. The flexible elongate member may pass through one of the pair of guide tubes to the article to be cut. The flexible elongate member may pass through the other of the pair of guide tubes from the article to be cut.

The guide tubes may be arranged adjacent each other, in which case, the flexible elongate member may pass substantially wholly around an object in said article to effect removal of said object during cutting of the article.

Alternatively, the guide tubes may be spaced from each other, in which case, the flexible elongate member may pass from one of said guide tubes, across an object in said article to be cut, to the other of said guide tubes

In another embodiment, the guide arrangement may comprise a single guide tube through which the flexible elongate member may pass to the article to be cut, and through which the flexible elongate member may pass from the article to be cut. In this embodiment, the flexible elongate member may extend from the guide tube to the article to be cut, around an object in said article, and from the article to be cut to return to the guide tube.

The guide arrangement may further comprise a guide rod to guide the flexible elongate member from the tensioning arrangement to the guide conduits. The guide rod may extend transverse to the guide conduits. The guide rod may define a recess, which may extend circumferentially of the guide rod, to receive the flexible elongate member.

The guide rod may define two guide recesses to receive the first and second passes of the flexible elongate member. Each guide recess may extend circumferentially around the guide rod. The recesses may be arranged to guide the flexible elongate member into the guide conduits.

The flexible elongate member may comprise a wire or a cable, and may be endless. Suitable abrasive formations may be provided on the wire or cable to facilitate cutting. The guide rod may define first and second guide recesses to receive the elongate member.

In one embodiment, the flexible elongate member may comprise a wire having cutting formations thereon, such as a Gigli wire. The flexible elongate member may comprise two or more wires, which may be twisted around each other. Cutting formations may be provided around the wires.

In an alternative embodiment, the flexible elongate member may comprise a wire. The flexible elongate member may include a coating of an abrasive material. In this embodiment, the wire may comprise steel, such as stainless steel. If desired, the wire may have a layer of another material thereon, between the wire and the abrasive material. Said other material may comprise a corrosion resistant material, such as copper.

In another alternative embodiment, the flexible elongate member may comprise wire formed of a shape memory alloy, such as nitinol or CuZnAl. The flexible elongate member may comprise a coating of an abrasive material.

In another embodiment still, the flexible elongate member may comprise a chain. The chain may have cutting members thereon. The cutting members may comprise twisted wire portions, or hooks. The cutting members may be wrapped around the chain. A plurality of cutting members may be provided along the chain. A respective cutting member may be provided on each link. Adjacent links may have cutting members provided on opposite sides. Alternatively, the chain has a coating of an abrasive material.

The abrasive material may be in the form of particles. The abrasive material may comprise diamond, tungsten and/or carbide.

In yet another alternative embodiment, the flexible elongate member may comprise a synthetic or plastics material, such as nylon.

In still another alternative embodiment, the flexible elongate member may comprise a plurality of strands, which may be wound around one another. In this embodiment, the flexible elongate member may comprise a Bowden wire.

In a further alternative embodiment, the flexible elongate member may be formed of woven filaments, wherein the filaments may be formed of a para-aramid synthetic fibre, such as Kevlar, a polyethylene material, which may be a high performance polyethylene material, such as Dynema.

In yet a further alternative embodiment, the flexible elongate member may comprise a spring, which may have a spring constant that is sufficiently high to minimise stretch during use, but which has flexibility. In this embodiment, the spring may have sharp edges to allow the flexible elongate member to saw through the material to be cut. The spring may have a profile which is rectangular or rhomboid in configuration.

In still a further alternative embodiment, the flexible elongate member may comprise a wrenched wire, which may be formed of steel. The wrenched wire may have a profile which is rectangular or rhomboid in configuration.

According to another aspect of this invention, there is provided a method of cutting an article comprising providing a cutting apparatus as described above, engaging the flexible elongate member with the article to be cut, and driving the carrying arrangement to feed the flexible elongate member and effect cutting of the article.

According to another aspect of this invention, there is provided a method of removing an object embedded in an article, comprising providing a cutting apparatus having a flexible elongate member, arranging the flexible elongate cutting member around the object, feeding the flexible elongate member in a lengthwise direction, and moving the wire through the article to cut the article around the object, thereby allowing the object to be removed from the article.

In one embodiment, the flexible elongate member may be fed from and to a single guide conduit, wherein the method may comprise forming a hole in the article adjacent the object.

The method may comprise moving the guide conduit into the hole while the flexible elongate member is fed through the conduit, thereby cutting the article.

Where the method comprises removing an object embedded in the article, the method may comprise arranging the flexible elongate member around the object and moving the guide conduit through the hole while feeding the flexible elongate member through the guide conduit to cut the article around the object. The step of moving the guide conduit through the hole may comprise pushing the cutting apparatus.

Alternatively, the method may comprise inserting the guide conduit into the hole, and thereafter arranging the flexible elongate member around the object. The method may then comprise removing the guide element from the hole while feeding the flexible elongate member through the guide element.

The step of arranging the flexible elongate member around the object may comprise attaching a first end of the flexible elongate member to a second end of the flexible elongate member. An attachment arrangement may be provided to attach the first and second ends to each other. The method may comprise forming a further hole transverse to the aforementioned hole to communicate with the aforementioned hole and facilitate the attachment of the first and second ends of the flexible elongate member to each other.

In another embodiment, the flexible elongate member may be fed from a first guide conduit and may be fed to a second guide conduit, wherein the method may comprise forming a first and second spaced holes in the article adjacent the object.

The method may comprise moving the first and second guide conduits into the first and second holes respectively hole while the flexible elongate member is fed through the conduits, thereby cutting the article.

Where the method comprises removing an object embedded in the article, the method may comprise arranging the flexible elongate member around the object and moving the first and second guide conduits through the first and second holes respectively while feeding the flexible elongate member through the first and second guide conduits to cut the article around the object. The step of moving the first and second guide conduits through the first and second holes respectively may comprise pushing the cutting apparatus.

Alternatively, the method may comprise inserting the first and second guide conduits into the first and second holes respectively, and thereafter arranging the flexible elongate member around the object. The method may then comprise removing the first and second guide elements from the first and second holes respectively while feeding the flexible elongate member through the guide element.

The step of arranging the flexible elongate member around the object may comprise attaching a first end of the flexible elongate member to a second end of the flexible elongate member. An attachment arrangement may be provided to attach the first and second ends to each other. The method may comprise forming a further hole transverse to the first hole to communicate with the first hole and facilitate the attachment of the first and second ends of the flexible elongate member to each other.

An embodiment of the invention will now be described by way of example only, with reference to the accompanying drawings, in which:

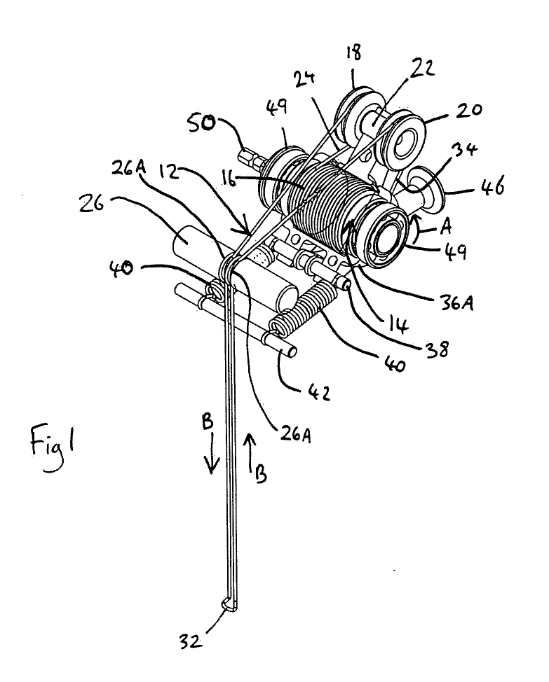

FIG. 1 is perspective view of the internal mechanism of a cutting apparatus;

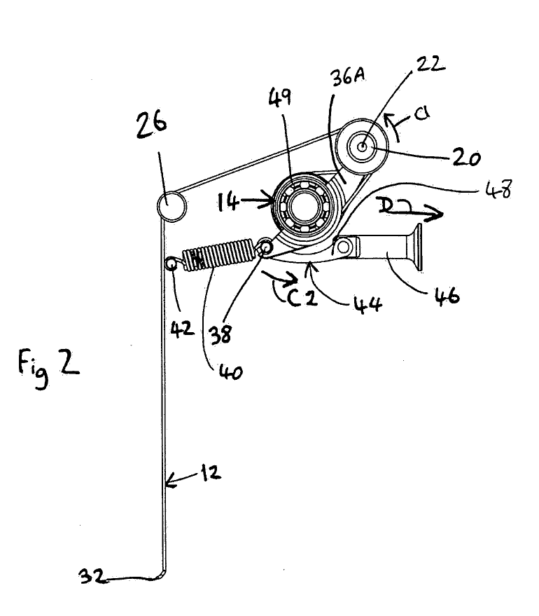

FIG. 2 is a side view of the internal mechanism shown in FIG. 1;

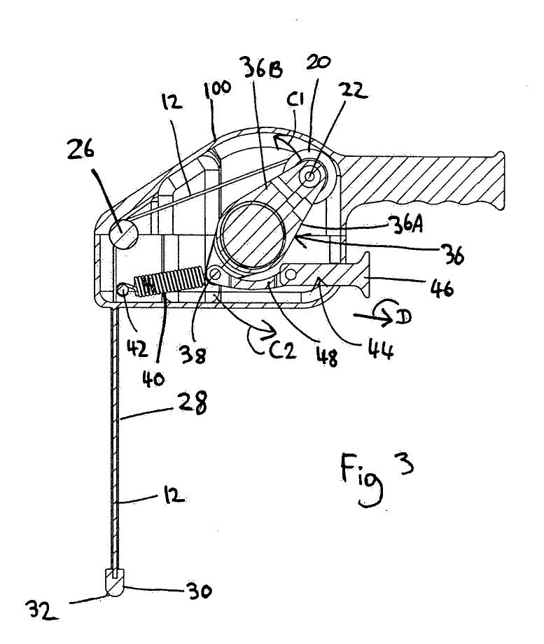

FIG. 3 is sectional side view of a cutting apparatus, showing a housing;

FIG. 4 is a side view of a cutting apparatus;

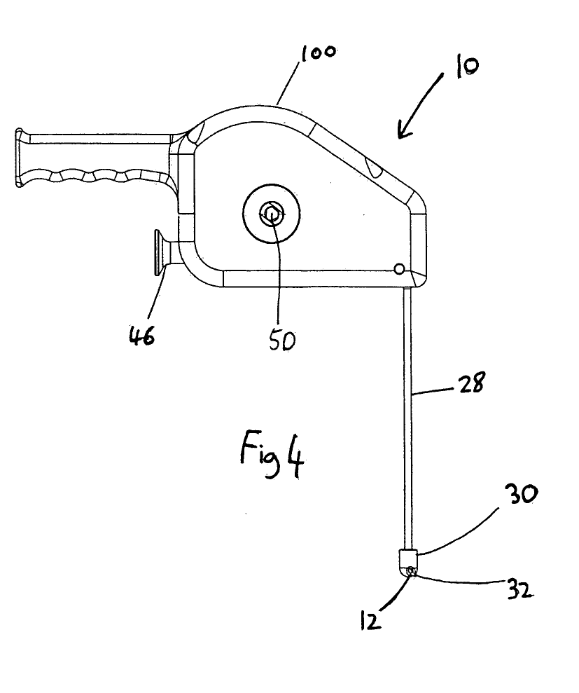

FIG. 5 is a side view of a flexible elongate member according to one embodiment;

FIG. 5A is a view along the lines V-V in FIG. 5;

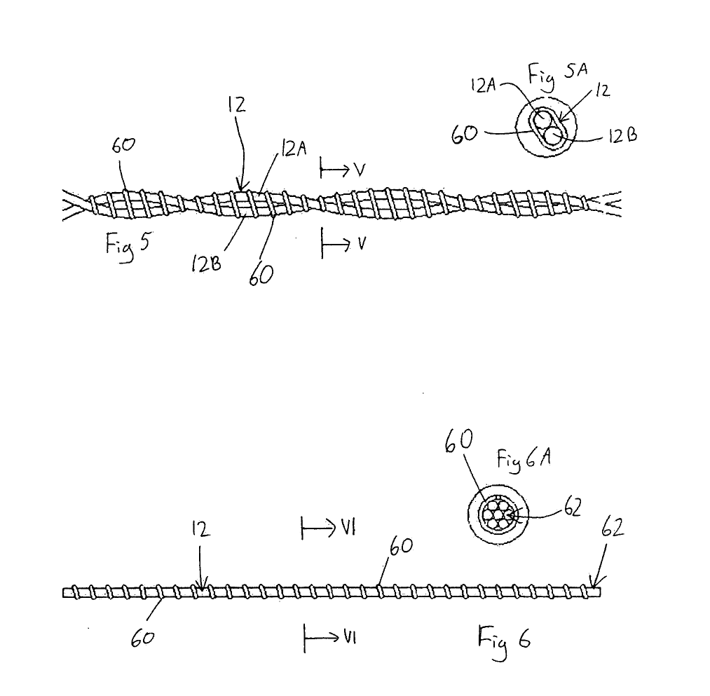

FIG. 6 is a side view of an alternative flexible elongate member;

FIG. 6A is a view along the lines VI-VI in FIG. 6;

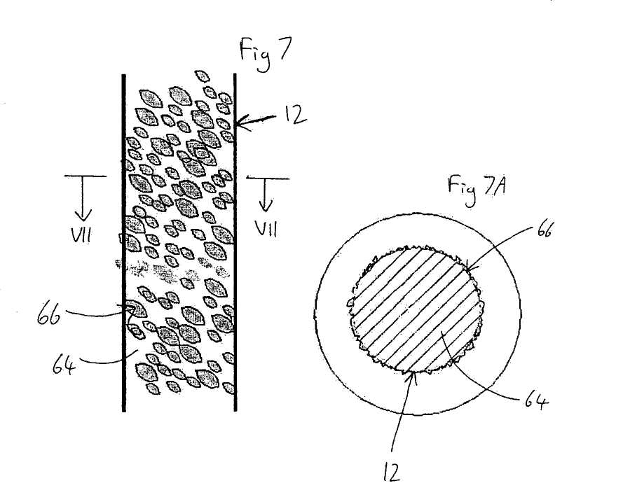

FIG. 7 is a side view of a flexible elongate member according to an alternative embodiment;

FIG. 7A is a view along the lines VII-VII in FIG. 7;

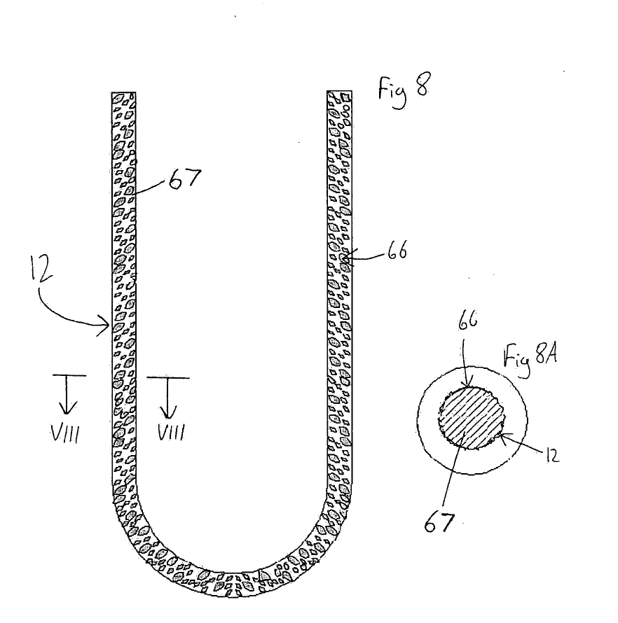

FIG. 8 is a side view of a flexible elongate member according to one embodiment;

FIG. 8A is a view along the lines VIII-VIII in FIG. 8;

FIG. 9 is a side view of a flexible elongate member according to one embodiment;

FIG. 9A is a view along the lines IX-IX in FIG. 9;

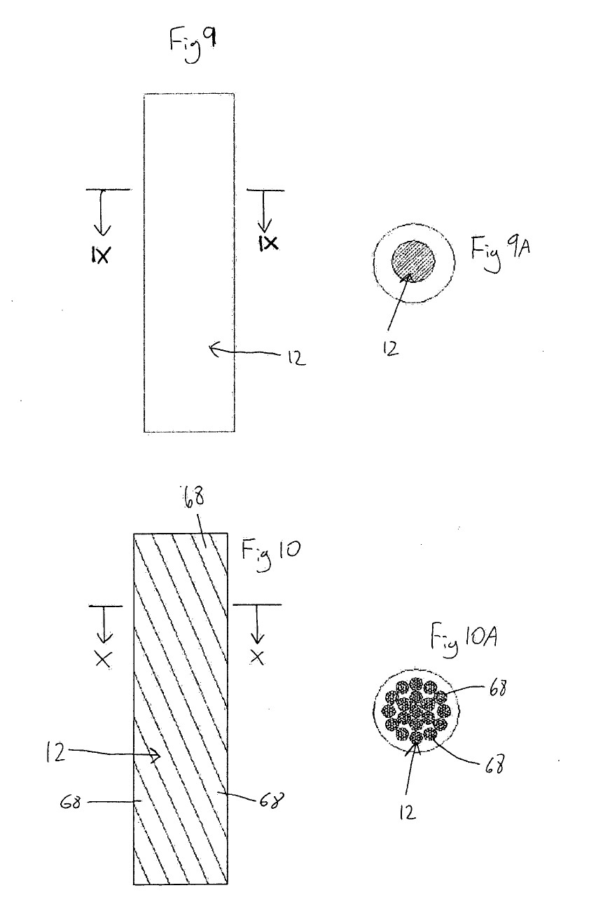

FIG. 10 is a side view of a flexible elongate member according to one embodiment;

FIG. 10A is a view along the lines X-X in FIG. 10;

FIG. 11 is a side view of a flexible elongate member according to one embodiment;

FIG. 11A is a view along the lines XI-XI in FIG. 11;

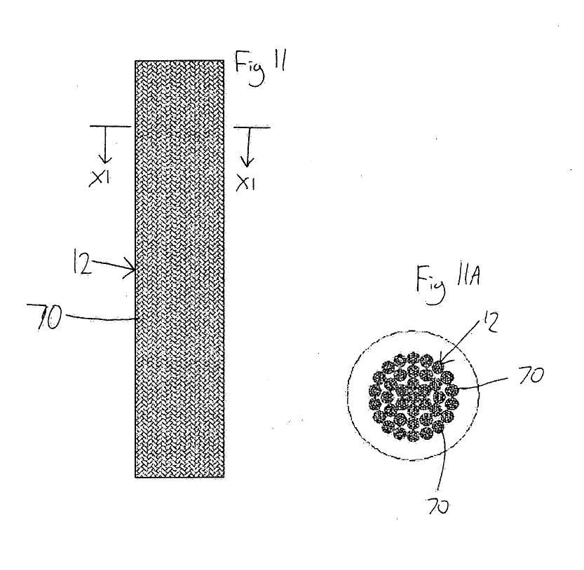

FIG. 12 is a side view of a flexible elongate member according to one embodiment;

FIG. 12A is a view along the lines XII-XII in FIG. 12;

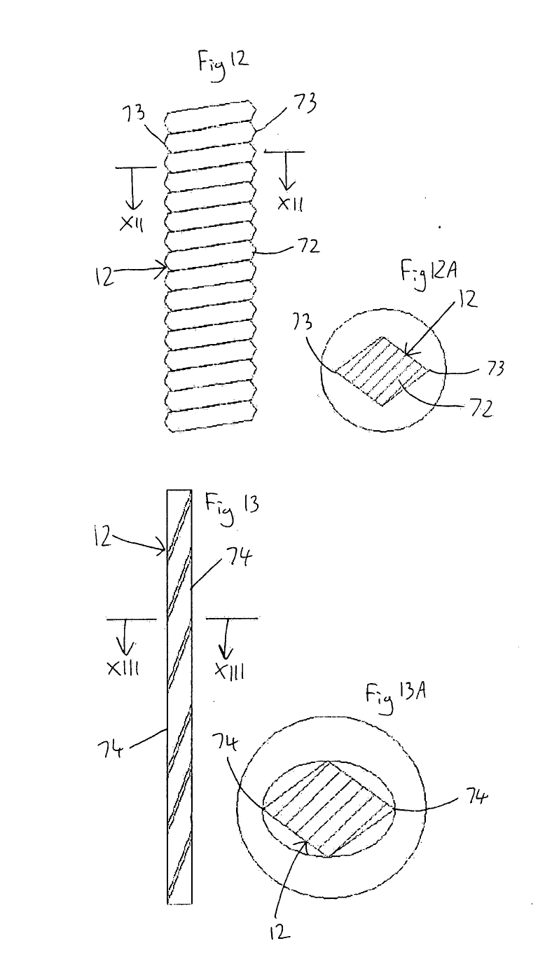

FIG. 13 is a side view of a flexible elongate member according to one embodiment;

FIG. 13A is a view along the lines XIII-XIII in FIG. 13;

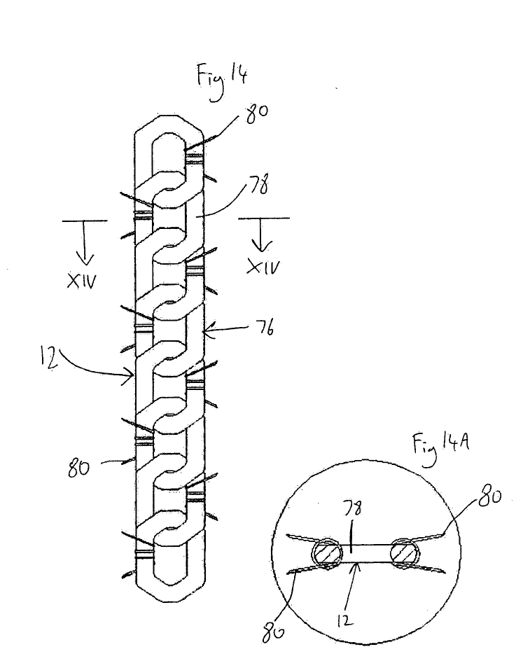

FIG. 14 is a side view of a flexible elongate member according to one embodiment;

FIG. 14A is a view along the lines XIV-XIV in FIG. 14;

FIG. 15 is a side view of a flexible elongate member according to one embodiment;

FIG. 15A is a view along the lines XV-XV in FIG. 15;

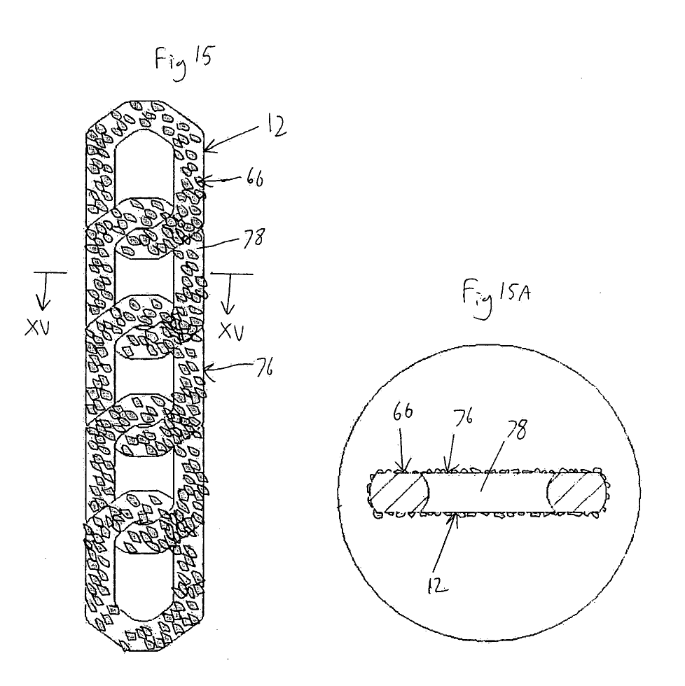

FIG. 16 shows an embodiment of a cutting apparatus in use;

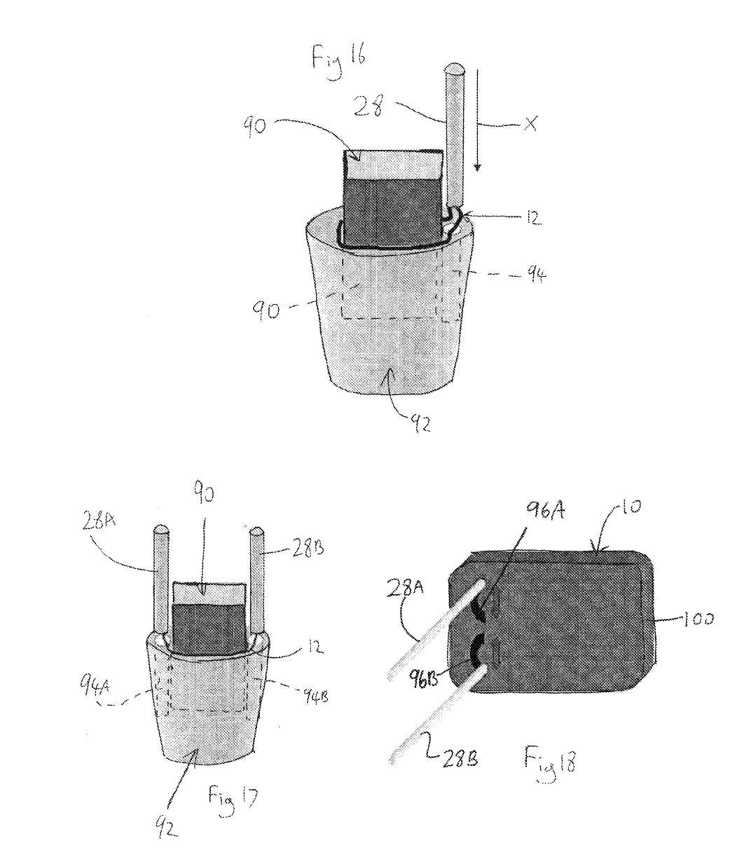

FIG. 17 shows another embodiment of a cutting apparatus in use;

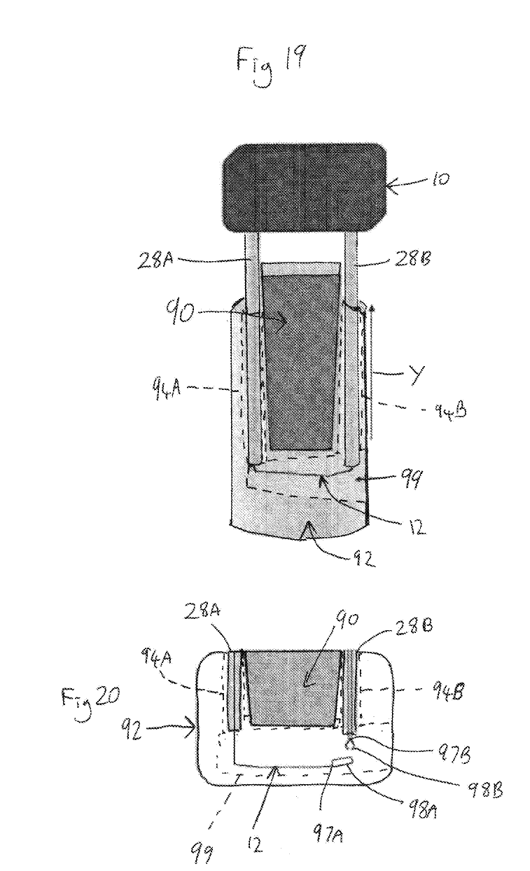

FIG. 18 shows the a bottom view of the cutting apparatus shown in FIG. 17;

FIG. 19 shows an alternative method of using the cutting apparatus shown in FIG. 17;

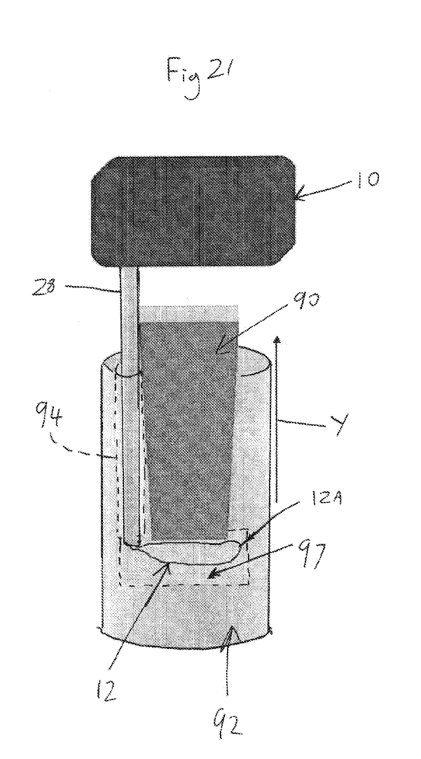

FIG. 20 shows the attachment of the ends of a flexible elongate member;

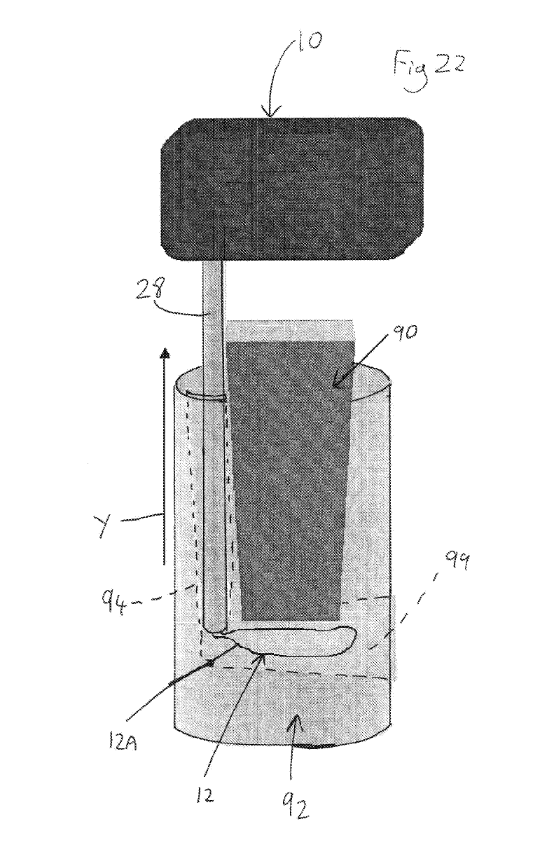

FIG. 21 shows a variation of a method of using the embodiment shown in FIG. 16; and

FIG. 22 shows a variation of the method shown in FIG. 21.

Referring to FIGS. 1 to 5 of the drawings, the internal mechanism of a cutting apparatus 10 is shown. The cutting apparatus 10 is suitable to be used to cut bone material or cement that surrounds a stem of, for example, an implant used in total hip arthroplasty.

The cutting apparatus 10 comprises a flexible elongate cutting member 12 in the form of a cutting wire, spring, cable or chain which, in the embodiment shown, is endless. The flexible elongate member 12 is arranged on a tensioning arrangement comprising a rotary member in the form of a drum 14. The flexible elongate member 12 is wound circumferentially around the drum 14 in a plurality of coils 16, so that the coils 16 of the flexible elongate member 12 extend adjacent one another across the drum 14.

Roller members in the form of first and second jockey wheels 18, 20 are provided adjacent the drum 14. The first and second jockey wheels 18, 20 are freely rotatable on an axle held in an axle sleeve 22. A first pass 24 of the flexible elongate member 12 extends from the drum 14 to the first jockey wheel 18 and then to a guide member 26.

The guide member 26 is cylindrical in configuration and has two circumferentially extending guide recesses 26A defined therein to receive the flexible elongate member 12.

The flexible elongate member 12 extends from one of the recesses 26A in the guide member 26 to one of two guide conduits, comprising two guide tubes in the form of cable housings 28 (see FIG. 4). The cable housings 28 comprise elongate tubular housings. The flexible elongate member 12 extends from the cable housing 28 to a nozzle 30. The recesses in the guide member 26 are positioned to guide the flexible elongate member 12 into the cable housings 28.

A loop 32 of the flexible elongate member 12 extends forwardly of the nozzle 30, and the flexible elongate member 12 extends from the loop 32 through the nozzle 30 and the other of the two cable housings 28 back to the guide member 26, at the other of the two recesses 26A. The nozzle 30 is provided to orient the flexible elongate member 12 as it passes from the cable housing 28.

The nozzle 30 provides directional changes in the flexible elongate member 12 that are not so sharp as to create excessive strain on the flexible elongate member 12, thereby reducing the necessary drive forces. The nozzle 30 also assists in preventing damage to the flexible elongate member 12. The flexible elongate member 12 extends from the guide member 26 to the second jockey wheel 20.

The flexible elongate member 12 extends around the second jockey wheel 20 to the drum 14 in a second pass 34. Thus, by rotating the drum in the direction indicated by the arrow A in FIG. 1, the flexible elongate member 12 moves as indicated by the arrows B in FIG. 1, wherein the flexible elongate member 12 is wound off the drum 14 in the first pass 24, and is wound onto the drum 14 in the second pass 34.

The first and second jockey wheels 18, 20 and the drum 14 are designed to hold the flexible elongate member 12 captive. This enables the tension in the flexible elongate member 12 to be released without the flexible elongate member 12 uncoiling on the drum 14 or moving off the first and second jockey wheels 12, 18.

As a result, the correct arrangement of the flexible elongate member 12 is maintained when the tension is reapplied.

It will, of course, be appreciated that the drum 14 could be rotated in the opposite direction to the arrow A, which would mean that the flexible elongate member 12 would move in the opposite direction to the arrow B.

Referring to FIG. 3, it can be seen that the drum 14 is held within a drum housing 36. The drum housing 36 is a close fit around the drum 14 to retain the coils 16 of the flexible elongate member 12 on the drum 14, and preventing the flexible elongate member 12 uncoiling itself from the drum 14 when the tension in the flexible elongate member 12 is released, as described below. The internal mechanism of the cutting apparatus 10, as shown in FIG. 1, and the drum housing 36 are held within an outer casing 100 (see FIGS. 3 and 4).

The drum housing 36 is provided with the first and second slots to guide the first and second passes 24, 34 of the flexible elongate member 12 into and out of the housing 36 and onto and off the drum 14.

The drum housing 36 provided in two parts, namely a lower part 36A and an upper part 36B. The upper and lower parts 36A and 36B hold the drum 14 between each other.

The housing 36 is pivotally movable about the drum 14 between a tension position shown in FIGS. 2 and 3, and a release position after pivotal movement of the housing 36 about the drum 14, as indicated by the arrows C1 and C2.

The jockey wheels 18, 20 are rotatably mounted on the housing 36. As a result, pivotal movement of the housing 36 in the direction of the arrows C1 and C2 also moves the first and second jockey wheels 18, 20 in the direction of the arrow C1 to the release position.

The movement of the jockey wheels 18, 20 in the direction indicated by the arrow C1 releases tension in the flexible elongate member 12, thereby allowing the flexible elongate member 12 to be paid out from the drum 14, with little or no flexible elongate member 12 being fed back on at the same time. The flexible elongate member 12 can then be looped around the article to be cut, such as the bone surrounding the stem of a femoral implant.

The opposite end of the drum housing 36 is connected to a first spring connecting bar 38, which is fixedly mounted on the lower part 36A of the drum housing 36. A pair of springs 40 extends from the first spring connecting bar 38 to a second spring connecting bar 42. The second connecting bar 42 is fixedly mounted on the casing 100.

A tension release means 44 is provided to open the drum housing 36 and release the tension in the flexible elongate member 12. The tension release means 44 comprises a manipulating member in the form of a handle 46 and a connecting lever 48. The connecting lever 48 is connected to the first spring connecting bar 38.

Referring to FIG. 3, the handle 46 can be pulled in the direction indicated by the arrow D. This causes the connecting lever 48 to pull on the first spring connecting bar 38 which, in turn, pivots the housing 36 in the direction indicated by the arrows C1 and C2 about the drum 14. This moves the first and second jockey wheels in the direction of the arrow C1 to the release position, thereby releasing tension in the flexible elongate member 12, as explained above.

The release of the tension in the flexible elongate member 12 allows the cable to the arranged around the stem of the implant so that the bone surrounding it can be cut.

When the handle 46 is released, the spring 40 pivots the housing 36 in the direction opposite to the arrows C1 and C2 to the tension position shown in FIGS. 2 and 3, thereby restoring the tension in the flexible elongate member 12.

The drum 14 is provided with bearings 49 at its opposite ends which engage the drum housing 36 to allow free rotation of the drum 14 within the drum housing 36. The drum 14 is provided at one of its end with a connection member 50 to connect the drum 14 to a suitable power tool (not shown) to provide power and rotate the drum 14.

In use, the tension in the flexible elongate member 12 is released by pulling the handle 46 in the direction indicated by the arrow D to pivot the drum housing 36 in the direction of the arrows C1 and C2. The loop 32 at the nozzle 30 can then be pulled forward and arranged across the stem of the implant to be extracted. The handle 46 is then released to close the drum housing 36 and apply tension again to the flexible elongate member 12.

A power tool is connected to the connecting member 50 and operated to rotate the drum in the direction indicated by the arrow A. This moves the flexible elongate member 12 in the direction indicated by arrow B so that the flexible elongate member 12 is wound off the drum at the first jockey wheel 18 and back onto the drum at the second jockey wheel 20.

The apparatus 10 is then pushed so that the loop 32 of the flexible elongate member 12, guided by the elongate tubular members 28, is moved along the stem to cut the bone material or cement adjacent the stem. This is continued until all the bone material or cement to which the stem is bonded has been removed. In this way, the stem is separated from the bone, and the implant can be removed.

It will be appreciated that, if desired, the power tool could rotate the drum in the direction opposite to the arrow A, in which case the flexible elongate member 12 leave in the direction opposite to the arrow B.

A suitable such flexible elongate member 12 for use with the above described embodiment of the invention is known as a Gigli wire, and is shown in FIG. 5.

The flexible elongate member 12 shown in FIGS. 5 and 5A comprises a pair of elongate strands 12A and 12B, which are twisted around each other in a generally helical manner. A plurality of cutting formations 60 are arranged around the strands 12A, 12B, and bind the strands 12A, 12B to each other. The cutting formations 60 are provided one after another in a spaced relationship along the lengths of the strands 12A, 12B.

In FIGS. 6 and 6A, the flexible elongate member 12 comprises a plurality of strands tightly wound around each other to provide a Bowden core 62. In embodiment shown, the Bowden core 60 comprises seven strands. A plurality of the cutting formations 60 are provided around the Bowden core 62.

In FIGS. 7 and 7A, the flexible elongate member 12 is formed of a single strand 64 of steel or stainless steel. The flexible elongate member 12 is coated with an abrasive material 66, in the form of particles of, for example, diamond, tungsten or carbide. Where the flexible elongate member 12 is not formed of stainless steel, it may be plated with a corrosion resistant material, such as copper.

In FIGS. 8 and 8A, the flexible elongate member 12 comprises a core 67 formed of a shape memory material, such as nitinol, or CuZnAl, and has a coating of particles of an abrasive material 66.

FIGS. 9 and 9A show a flexible elongate member 12 formed of a single strand nylon or steel.

FIGS. 10 and 10A show a flexible elongate member 12 formed of a plurality of strands 68 of stainless steel or plated steel. The strands 68 are tightly helically wound around each other so that the flexible elongate member 12 is in the form of a Bowden wire.

Another embodiment is shown in FIGS. 11 and 11A, in which the flexible elongate member 12 is formed of woven strands 70 of a high strength synthetic material, such as para-aramid fibres, for example Kevlar, or high performance polyethylene fibres, for example Dynema.

FIGS. 12 and 12A show a flexible elongate member 12 in the form of a spring 72, having a high spring constant, thereby minimising stretch. The edges 73 of the spring are sharp allowing it to cut. As can be seen from FIG. 12A, the cross-sectional profile of the flexible elongate member is rhomboid.

FIGS. 13 and 13A show a flexible elongate member 12, formed of a wrenched steel wire, which has a rhomboidal cross-sectional profile, with sharp edges 74

FIGS. 14 and 14A show a flexible elongate member 12 in the form of a chain 76, having a plurality of chain links 78. Cutting members 80, in the form of twisted wire portions or hooks, may be provided along the chain. Each link 78 may be provided with a respective cutting member 80, so that adjacent links have cutting members 80 on respective opposite sides.

As an alternative to the cutting members 80, FIGS. 15 and 15A show an embodiment, in which the chain 76 is coated with an abrasive material 66, which can be the same as the abrasive material described above.

FIGS. 16 to 21 show embodiments of the cutting apparatus 10 in use in removing an object 90 from an article 92. In one use of the cutting apparatus 10, the object 90 may comprise a stem of a bone implant, and the article 92 may comprise a bone. In other uses of the cutting apparatus 10, the object 90 may comprise any other type of object 90 implanted in any other type of article 92. For example, the object 90 could be a nail implanted into an article in the form of concrete.

In FIG. 16, a single cable housing 28 is shown through which the flexible elongate member 12 passes in both directions. Initially, a hole 94 is drilled into the article 92 to the depth beyond which the object 90 extends into the article 92. The flexible elongate member 12 is arranged around the object 90, as shown in FIG. 16.

The cutting apparatus 10 is then turned on to drive the flexible elongate member 12, and the cutting apparatus 10 is manipulated to push the cable housing 28 into the hole 94, as indicated by the arrow X. As the cable housing 28 is pushed into the hole 94, the flexible elongate member 12 cuts the article 92 around the object 90, so that when the cable housing 28 reaches the end of the hole 94, the object 90 has been cut free from the article 92 and can be removed.

In FIG. 17, first and second cable housing is 28A and 28B are provided on the cutting apparatus 10. The flexible elongate member 12 passes out of the cable housing 28A and into the cable housing 28B. It will be appreciated that, if desired, the flexible elongate member 12 could pass out of the cable housing 28B and into the cable housing 28A.

First and second holes 94A and 94B are drilled into the article 92 on either side of the object 90 to a depth beyond the depth of the object 90 in the article 92. As can be seen from FIG. 17, the flexible view on date member 12 extends across the object 90 on one side thereof.

The cutting apparatus 10 is turned on to drive the football elongate member and the cutting apparatus 10 is manipulated to push the first and second cable housings 28A and 28B into the first and second holes 94A and 94B respectively. The movement of the first and second cable housings 28A and 28B into the first and second holes 94A and 94B causes the flexible elongate member to cut the article 92 around the object 90.

When the first and second cable housings 28A and 28B reach the ends of the first and second holes 94A and 94B, and they are removed from the first and second holes 94A and 94B. Cutting apparatus 10 is then turned round so that the first cable housing 28A is inserted into the second hole 94B, and the second cable housing 28B is inserted into the first hole 94A. In this orientation, the flexible elongate member 12 extends across the object 90 on the opposite side thereof.

When the cutting apparatus 10 is turned on and the first and second cable housings 28A and 28B are pushed into the second and first holes 94B and 94A, the article 92 on the other side of the object 90 is cut. When the first and second cable housings 28A and 28B reach the ends of the second and first holes 94B and 94A, the object 90 can be removed from the article 92.

FIG. 18 shows the view from below of the cutting apparatus 10 which it can be seen that the first and second cable housings 28A and 28B extend into the outer casing 100 via elongate curved slots 96A and 96B. the elongate curved slots 96A and 96B allow the position of the first and second cable housings 28A and 28B to be adjusted relative to each other in accordance with the size of the object 90.

Referring to FIGS. 19 and 20, there is shown an alternative way of using the cutting apparatus 10 shown in FIG. 17. As can be seen from FIG. 20, the flexible elongate member 12 has first and second ends 97A and 97B, which are provided with an attachment of arrangement in the form of the first attachment formation 98A on the first end 97A, and a second attachment formation 98B on the second end 97B. The first and second attachment formations 98A and 98B are configured to cooperate with each other to attach the first and second ends 97A and 97B of the flexible elongate member 12 together.

In order to facilitate the attaching of the first and second ends 97A and 97B to each other, a lateral hole 99 is drilled into the side of the article 92 to communicate with the first and second holes 94A and 94B. This allows with the first and second ends 97A and 97B to be manipulated to be attached together.

The cutting apparatus 10 can then be turned on to drive the flexible elongate member 12. The cutting apparatus 10 can then be pulled upwardly, and shown by the arrow Y to cut the article 92 on one side of the object 90.

After the first and second cable housings 28A and 28B are removed from the first and second holes, 94A and 94B the cutting apparatus 10 is turned round. The second cable housing 28B is then inserted into the first hole 94A, and the first cable housing 28A is inserted into the second hole 94B.

The above steps are then repeated to attach the first and second ends 97A and 97B of the flexible elongate member 12 to each other, and to remove the first and second cable housings 28A and 28B from the second and first holes 94B and 94A while the flexible elongate member is driven to cut the article 92 on the other side of the object 90. The object 90 can then be removed from the article 92.

FIG. 21 shows another way of using the cutting apparatus 10 shown in FIG. 16, which is similar to the way described above for FIGS. 19 and 20. In FIG. 21, a cavity 97 is formed in the article 92 at the end of the hole 94, for example by reaming out the region of the article 92 beyond the end of the object 90. This allows the single cable housing 28 to be inserted into the hole 94 and a flexible elongate member 12 arranged in a position where it can extend around the object 90. for this purpose, the flexible elongate member 12 can be formed of a shape memory material, such as shown in FIGS. 8 and 8A, so that the flexible elongate member 12 forms a loop 12A to allow it to be arranged over the end of the object 90. X-ray monitoring may be required to ensure that the loop 12A formed by the flexible elongate member 12 fits around the end of the object 90.

FIG. 22 shows another way of using the cutting apparatus 10 shown in FIG. 21, which is similar to the way described above for FIG. 21. However, in FIG. 22, a lateral hole 99 is drilled into the article 92 at the end of the hole 94. This allows the single cable housing 28 to be inserted into the hole 94 and a flexible elongate member 12 arranged in a loop 12A so that it can extend around the object 90.

When the flexible elongate member 12 is arranged as shown in FIGS. 21 and 22, the cutting apparatus 10 can be turned on and the cable housing 28 pulled out of the hole 94 in the direction of the arrow Y. This causes the flexible elongate member 12 to move upwardly around the object 90, thereby cutting the article 92, so that the object 90 can be removed from the article 92.

There is thus described a cutting apparatus which provides an effective means for cutting an object embedded in an article. In one example, the cutting apparatus 10 is particularly suitable for cutting the cement or bone material around a stem of a bone implant, thereby enabling the implant to be removed.

Various modifications can be made without departing from the scope of the invention.

1. A cutting apparatus comprising a flexible elongate cutting member, and a tensioning arrangement to maintain the flexible elongate member in a tensioned condition, the tensioning arrangement comprising a carrying arrangement on which the flexible elongate member is arranged, and the cutting apparatus further including a driving arrangement to move the carrying arrangement, thereby causing the flexible elongate member to move and effect cutting of an article.

2. A cutting apparatus according to claim 1, wherein the carrying arrangement comprises a rotary arrangement, and the flexible elongate member is wound around the rotary arrangement.

3. A cutting apparatus according to claim 2, wherein the rotary arrangement comprises a rotary member and at least one bearing on which the rotary member is mounted to allow rotation of the rotary member.

4. A cutting apparatus according to claim 1, wherein the driving arrangement comprises a connecting portion configured to be connected to a prime mover.

5. A cutting apparatus according to claim 1, wherein the tensioning arrangement further comprises at least one roller member, around which the elongate member passes.

6. A cutting apparatus according to claim 5, wherein the roller member is movable between a tension position in which the flexible elongate member is held in tension, and a release position, in which the tension in the flexible elongate member is relaxed, and when the roller member is in the release position, a desired length of the flexible elongate member can be fed from the rotary arrangement and a length less than said desired length can be fed onto the rotary arrangement at the same time.

7. A cutting apparatus according to claim 6, wherein the tensioning arrangement comprises urging means to urge the roller member to the tension position.

8. A cutting apparatus according to claim 7, comprising a release means to move the roller member to the release position, the release means being pivotally mounted on the housing to pivot the housing about the rotary member to the release position.

9. A cutting apparatus according to claim 7, wherein the release means comprises a manipulating element and a connecting lever pivotally attached to the housing.

10. A cutting apparatus according to claim 9, wherein the connecting lever is attached to the connecting rod, and the connecting rod connects the connecting lever to the housing, and wherein the connecting lever is pivotally connected to the manipulating element.

11. A cutting apparatus according to claim 1, wherein the tensioning arrangement further comprises a holding arrangement around the rotary member to hold the flexible elongate member on the rotary member.

12. A cutting apparatus according to claim 11, wherein the holding arrangement comprises a housing closely spaced from the rotary member to allow rotation of the rotary member relative thereto, and said housing being close enough to the rotary member to prevent uncoiling of the flexible elongate member from the rotary member when the roller members are in the release position.

13. A cutting apparatus according to claim 11, wherein the holding arrangement defines first and second apertures to allow the elongate member to be fed to or from the drum.

14. A cutting apparatus according to claim 1, comprising a guide arrangement to guide the flexible elongate member, the guide arrangement comprising at least one guide conduit, the flexible elongate member extending through the guide conduit.

15. A cutting apparatus according to claim 14, wherein the guide conduit comprises at least one guide tube.

16. A cutting apparatus according to claim 14, wherein the guide conduit is formed of a rigid material to allow the elongate member to be pushed into the material to be cut.

17. A cutting apparatus according to claim 15, wherein the guide arrangement comprises a pair of guide conduits, the flexible elongate member passing through one of the pair of guide conduits to the article to be cut, and passing through the other of the pair of guide conduits from the article to be cut.

18. A cutting apparatus according to claim 17, wherein the guide conduits are arranged adjacent each other, and the flexible elongate member passes substantially wholly around an object in said article to effect removal of said object during cutting of the article.

19. A cutting apparatus according to claim 17, wherein the guide conduits are spaced from each other, and the flexible elongate member can be arranged to pass from one of said guide conduits, across an object in said article to be cut, to the other of said guide conduits.

20. A cutting apparatus according to claim 15, wherein the guide arrangement comprises a single guide conduit through which the flexible elongate member passes to the article to be cut, and through which the flexible elongate member passes from the article to be cut.

21. A cutting apparatus according to claim 14, wherein the guide arrangement further comprises a guide rod to guide the flexible elongate member from the tensioning arrangement to the, or each, guide conduit, the guide rod extending transverse to the, or each, guide conduit.

22. A cutting apparatus according to claim 1, wherein the flexible elongate member comprises a wire or a cable, and abrasive formations are provided on the wire or cable to facilitate cutting.

23. A method of removing an object embedded in an article, comprising providing a cutting apparatus having a flexible elongate member, arranging the flexible elongate cutting member around the object, feeding the flexible elongate member in a lengthwise direction, and moving the wire through the article to cut the article around the object, thereby allowing the object to be removed from the article.

24. A method according to claim 24, wherein the flexible elongate member is fed from and to a single guide conduit, and the method comprises forming a hole in the article adjacent the object.

25. A method according to claim 24, comprising moving the guide conduit into the hole while the flexible elongate member is fed through the conduit, thereby cutting the article.

26. A method according to claim 25, comprising arranging the flexible elongate member around the object and moving the guide conduit through the hole while feeding the flexible elongate member through the guide conduit to cut the article around the object.

27. A method according to claim 26, wherein the step of moving the guide conduit through the hole comprises pushing the cutting apparatus.

28. A method according to claim 25, comprising inserting the guide conduit into the hole, arranging the flexible elongate member around the object, and thereafter pulling the guide element from the hole while feeding the flexible elongate member through the guide element.

29. A method according to claim 25, comprising feeding the flexible elongate member from a first guide conduit to a second guide conduit, wherein the method comprises forming a first and second spaced holes in the article adjacent the object.

30. A method according to claim 29 comprising moving the first and second guide conduits into the first and second holes respectively while the flexible elongate member is fed through the conduits, thereby cutting the article.

31. A method according to claim 30, comprising arranging the flexible elongate member across the object and moving the first and second guide conduits through the first and second holes respectively while feeding the flexible elongate member through the first and second guide conduits to cut the article around the object.

32. A method according to claim 31, wherein the step of moving the first and second guide conduits through the first and second holes respectively comprises pushing the cutting apparatus.

33. A method according to claim 29, comprising inserting the first and second guide conduits into the first and second holes respectively, and thereafter arranging the flexible elongate member around the object.

34. A method according to claim 33, wherein after the step of arranging the flexible elongate member around the object, the method comprises removing the first and second guide elements from the first and second holes respectively while feeding the flexible elongate member through the guide element.

35. A method according to claim 34, wherein the step of arranging the flexible elongate member around the object comprises attaching a first end of the flexible elongate member to a second end of the flexible elongate member.

36. A method according to claim 35, wherein an attachment arrangement is provided to attach the first and second ends to each other, and the method comprises forming a further hole transverse to the first hole to communicate with the first hole and facilitate the attachment of the first and second ends of the flexible elongate member to each other.
